(19) United States
(12) Patent Application Publication
(10) Pub. No.: US 2007/0293813 A1
(43) Pub. Date: Dec. 20, 2007

(54) INTERNAL OSSEOUS DELIVERY SYSTEM AND METHOD

(76) Inventor: David B. Spenciner, North Attleboro, MA (US)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 11/625,951

(22) Filed: Jan. 23, 2007

Related U.S. Application Data
(60) Provisional application No. 60/804,975, filed on Jun. 16, 2006.

(51) Int. Cl. A61M 3/00

(52) U.S. Cl. 604/43

(57) ABSTRACT

A system for delivering a therapeutic agent including an inflow pathway and an outflow pathway coupled to osseous tissue. The inflow pathway is coupled to a source of therapeutic agent to be delivered to the osseous tissue. The outflow pathway allows the removal of material from the osseous tissue. A pump is connected to at least one of the inflow pathway and the outflow pathway to facilitate delivery of the therapeutic agent to the osseous tissue or removal of material from the osseous tissue.

### CROSS-REFERENCE TO RELATED APPLICATIONS

This application claims the benefit of U.S. provisional patent application Ser. No. 60/804,975, filed Jun. 16, 2006. The entire disclosure of which is incorporated herein by reference.

### FIELD

The present disclosure generally relates to a system for the delivery of agents to a patient, and more particularly to an inter-osseous delivery system.

### BACKGROUND

Treatment of various bone diseases and afflictions may often require the delivery of a medicament to the region of the afflicted bone. Conventionally, delivery of the medicament may be accomplished by creating a passage into the bone to the point of interest. The medicament may then be injected into the bone, e.g., via a cannula, needle, or the like. Such a procedure may often require directly accessing the point of interest, e.g., by drilling a passage into the bone directly to the point of interest. Additionally, depending upon the quantity of medication delivered, the procedure may result in an undesirable increase in pressure within the bone.

### BRIEF DESCRIPTION OF THE DRAWINGS

Features and advantages of the present invention are set forth by way of description of exemplary embodiments consistent therewith, which description should be considered in conjunction with the accompanying drawings, wherein:

FIG. 1 schematically illustrates an embodiment of a system for osseous delivery of an agent to a vertebral body;

FIG. 2 schematically illustrates an embodiment of a system for osseous delivery of an agent to a femoral head;

FIG. 3 schematically illustrates an embodiment of a system providing reversible flow direction for osseous delivery of agents to a skull;

FIG. 4 schematically illustrates an embodiment of an osseous delivery system for delivering components of a two part cement to a fracture within a bone; and

FIG. 5 schematically illustrates an embodiment of an osseous delivery system for sequentially delivering multiple therapeutic agents.

### DESCRIPTION

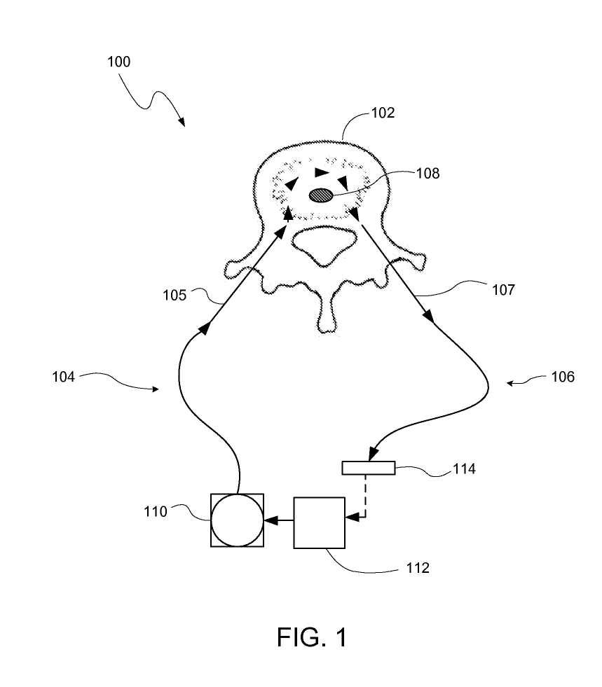

FIG. 1 illustrates an embodiment of an osseous delivery system 100 which may be used to infuse and/or deliver a therapeutic agent to a patient. The therapeutic agent may be delivered to the patient by delivery into osseous tissue, including, for example bone, cartilaginous structure or tissue, etc., such as a vertebra 102. The therapeutic agent may be delivered into the vertebra 102 via an inflow pathway 104. In addition to delivering the therapeutic agent into the vertebra 102, material may be removed from the osseous tissue through an outflow pathway 106.

In the context of the present disclosure, a therapeutic agent may include any material which may be delivered to a patient during the course of a diagnostic, therapeutic, or other procedure. In one example, the therapeutic agent may be an anti-tumor agent which may be provided for the treatment of a tumor 108 in the vertebra 102. The osseous delivery system 100 may deliver the anti-tumor agent to the region of the tumor 108 within the vertebra 102. The anti-tumor agent need not be delivered directly to the tumor 108. In one embodiment, the anti-tumor agent may be delivered to the vertebra 102 generally, and may reach the tumor 108 as needed through diffusion and/or migration of the anti-tumor agent trough the osseous tissue.

Various mechanisms may be involved in the transport or movement of a therapeutic agent and/or carrier through osseous tissue. In one embodiment, the inflow pathway 104 and the outflow pathway 106 may be directly connected. For example, a tunnel or passage may be formed through the osseous tissue extending at least a portion of the way between the inflow pathway 104 and the outflow pathway 106. The tunnel or passage may facilitate the flow of fluids between the inflow pathway 104 and the outflow pathway 106 and to the tumor 108. For example, a suitable tunnel may be provided by drilling a tunnel between the inflow pathway 104 and the outflow pathway 106. In further embodiments, the inflow pathway 104 and the outflow pathway 106 may be indirectly connected. An example of indirect connection between the inflow pathway 104 and the outflow pathway 106 may include connection via open trabecular structure or naturally occurring passages through the osseous tissue.

The material removed from the vertebra via the outflow pathway 106 may include the anti-tumor agent and/or other material delivered to the vertebra 102. Furthermore, the material removed from the vertebra may include fluids, such as bodily fluids, etc., particulate material, tissue, etc. residing in the vertebra 102. The removal of material from the vertebra 102 may at least partially offset the volume of material delivered to the vertebra. In this manner, it may be possible to prevent and/or control an undesired build up of anti-tumor agent and/or carrier for the anti-tumor agent within the vertebra 102. Still further, the outflow passage 106 may allow the removal of at least a portion of one or more byproducts which may result from the delivery of the anti-tumor agent to the vertebra. Additionally, and/or alternatively the outflow passage 106 may allow the removal of material to prevent and/or control an increase in interosseous pressure, which may result from the deliver of the anti-tumor agent to the vertebra 102.

The inflow and outflow pathways 104, 106 may include cannulas 105, 107 inserted into openings, or portals, in the vertebra 102. The openings may include holes drilled into the vertebra 102 allowing the cannulas 105, 107 to be positioned at least partially within the vertebra 102, for example in the general region of a tumor 108 within the vertebra 102. The inflow pathway 104 may be coupled to a pump 110. The pump 110 may, in turn, be coupled to a supply 112 of the anti-tumor agent, such as a reservoir, etc. The therapeutic agent may be delivered from the supply 112 to the vertebra 102 via the pump 110 and through the inflow pathway 104.

In another embodiment, rather than providing the pump connected to a reservoir of the anti-tumor agent, the delivery system may operate in an at least generally closed loop. For example, the pump may be coupled to the outflow pathway. After the introduction of an initial quantity of the anti-tumor agent, the continued delivery anti-tumor agent may depend upon material extracted from the osseous tissue. That is, after the initial charge of anti-tumor agent, subsequent delivery of anti-tumor agent may depend, at least in part, upon the removal of material, e.g., removal of the anti-tumor agent, from the osseous tissue via the outflow pathway. Such a system may include a buffer reservoir to provide a sufficient supply of anti-tumor agent to overcome any time lag between the deliver of the anti-tumor agent and the subsequent removal of material from the osseous tissue.

As shown in FIG. 1, the osseous delivery system 100 may be configured to re-circulate the material removed from the vertebra 102. The outflow pathway 106 may be coupled to the supply 112 of therapeutic agent. The material removed from the vertebra 102 via the outflow pathway 106 may be pumped back to the vertebra 102 via the pump 110 and the inflow pathway 104. In other embodiments, the material removed from the osseous tissue may not be re-circulated. In such an embodiment, the material removed from the osseous tissue may be separately collected and/or discarded, thereby providing an open-loop system

As noted, a time lag may exist between the delivery of the therapeutic agent to the osseous tissue and the removal of material from the osseous tissue. In an embodiment in which the material removed from the osseous tissue includes the therapeutic agent, at least a portion of the time lag may result from the time required for the diffusion and/or migration of the therapeutic agent between the inflow pathway 104 and the outflow pathway 106. In other embodiments, the material removed from the osseous tissue may not, however, include and/or exclusively include the therapeutic agent. In such a circumstance, the time lag between the delivery of the therapeutic agent and the removal of material from the osseous tissue may not be dependent upon rate of diffusion and/or migration of the therapeutic agent through the osseous tissue. In further embodiments, there may be little or no time lag between the delivery of a therapeutic agent to the osseous tissue and the removal of material from the osseous tissue. That is, removal of material may occur nearly simultaneously with the delivery of therapeutic agent.

During the course of a delivery procedure of the therapeutic agent to the osseous tissue, the delivery of the therapeutic agent and the removal of material may achieve a steady state condition. That is, even in the event of an initial time lag between the delivery of the therapeutic agent and the removal of material, for a constant rate of delivery of a therapeutic agent, a constant rate of removal of material from the osseous tissue may be achieved. The constant removal rate may, in some embodiments, differ from the constant delivery rate.

As mentioned, in some embodiments, the rate of removal of material from the osseous tissue may differ from the rate of delivery of a therapeutic agent to the osseous tissue. For example, at least a portion of the therapeutic agent delivered to the osseous tissue may be absorbed by the patient, resulting in a relatively lower rate of material removed from the osseous tissue. Conversely, the therapeutic agent may create a byproduct and/or may stimulate the release of material from the patient. In such an embodiment, the rate of removal of material from the osseous tissue may exceed the rate of delivery of the therapeutic agent. In still further embodiments, the removal of material from the osseous tissue may not be exclusively related to the delivery and/or rate of delivery of the therapeutic agent, e.g., when removal of material is related to relieving interosseous pressure, etc. In some such embodiments no steady state condition may be achieved.

The rate of delivery of the therapeutic agent and/or the rate of removal of material from the osseous tissue may remain constant for the duration procedure. Alternatively, rate of removal and/or the rate of delivery may vary during the course of the procedure. Variations in the delivery and/or removal rates may be the result in changes in the uptake or release rate of the osseous tissue. Variations may also be a result of predetermined scheme. For example, the delivery and/or removal rate may follow a ramp-up or ramp-down scheme and/or may vary according to a cyclic protocol.

The osseous delivery system may include additional features disposed between inflow and outflow pathways. For example, in the context of a re-circulating system, one or more filters 114 may be provided between the outflow pathway 106 and the inflow pathway 104 to remove particulate matter, contaminants, etc. from the therapeutic agent prior to delivering the therapeutic agent to the vertebra. Suitable filters may be configured to provide desired removal or separation. For example, a filter may be provided to remove particulate debris from the removed material. Other filters may provide the separation and/or removal of solid and/or liquid and/or gaseous components. Additionally, a filter may provide physical and/or chemical conversion and/or modification of one or more components in the outflow material.

Facility for testing the therapeutic agent and/or the material removed from the vertebra, i.e., the outflow material, may also be provided. The outflow may be tested for presence and/or the concentration of various materials and/or components, etc. Testing of the outflow may be accomplished in an in-line manner, or by the provision for removing specimens from the outflow. Testing and/or the collection of specimens may occur either before or after filtration, if any. Similarly, testing of materials separated and/or extracted through a filtration operation may also be carried out.

An osseous delivery system may allow additional material, e.g., therapeutic agents, etc., to be added and/or controlled prior to delivery. In an embodiment in which the outflow may be re-circulated, the addition of material to the outflow may allow a concentration of the therapeutic agent, e.g., the anti-tumor agent, or other component of the delivered material, to be maintained within a desired range, etc. That is, the outflow may be fortified, e.g., with the anti-tumor agent, prior to re-delivery in order to maintain desired concentrations of the anti-tumor agent delivered to the patient. In this manner, the concentration of the agent may be compensated for any portion lost, e.g., due to uptake by the patient.

In related embodiments, the content and/or concentration of one or more component to be delivered to the patient may be dynamically controlled. That is, the agents and/or concentration of agents to be delivered to the patient may be controlled and/or adjusted throughout the course of the procedure. Control and/or adjustment of the content and/or concentration of agents may be based on an analysis of the inflow, the outflow and/or another tested and/or evaluated quantity, e.g., patient response, etc. Additionally, and/or alternatively, control and/or adjustment of the agents may be made according to schedule or schema, e.g., a ramp-up or cycling of concentration, etc. Consistent with any of the foregoing aspects, control and/or adjustment of the content and/or concentration of agents may be carried out either continuously or intermittently. The addition of material may be made to the outflow of material from the osseous tissue, to the reservoir, or to the inflow of material being delivered to the osseous tissue.

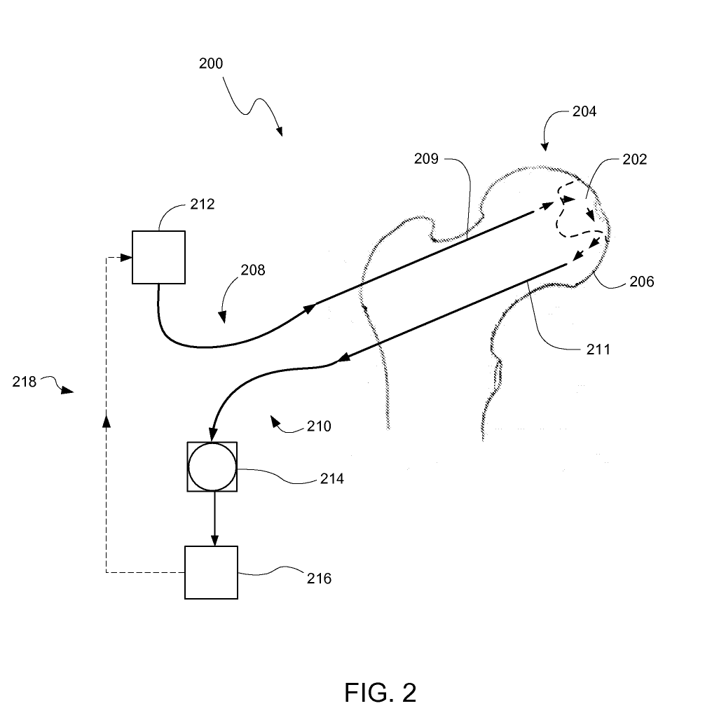

Turning to FIG. 2, another embodiment of an osseous delivery system 200 is shown configured for the treatment of a bone edema 202, e.g., of the head 204 of a femur 206. The osseous delivery system 200 may include an inflow pathway 208 and an outflow pathway 210. The inflow and outflow pathways 208, 210 may include respective cannulas 209, 211 extending at least partially into the femur 206. As depicted, the inflow and outflow cannulas 209, 211 may be positioned within the femoral head 204 adjacent to the region of the edema 202.

The inflow pathway 208 may be coupled to a supply 212 of a blood-thinning agent, or other medicament, etc., which may be used a part of a treatment regime for the bone edema 202. The inflow pathway 208 may, therefore, allow the delivery of the blood-thinning agent from the supply 212 to the region of the edema 202 via the inflow cannula 209. Other therapeutic agents may, of course, also be used in connection with the treatment.

The outflow pathway 210 may be coupled to a pump 214, which may provide reduced pressure, e.g., suction, in the outflow pathway 210. The reduced pressure provided in the outflow pathway 210 may facilitate removal of material from the femur 206, such as in the region of the edema 202 of the femoral head 204. The outflow pathway 210 may further be coupled to a collection container 216 for receiving material removed via the outflow pathway 210.

The osseous delivery system 200 may provide both the delivery of the blood-thinning agent through the inflow pathway 208 and the removal of material through the outflow pathway 210. The low pressure on the outflow pathway 210 and/or gravitational pressure on the supply 212 of blood-thinning agent may facilitate delivery of the blood-thinning agent to the femur 206. Similarly, the presence of the outflow pathway 210, and/or the low pressure on the outflow pathway 210 provided by the pump 214, may facilitate removal of material from the femur 206. In a further embodiment, the use of a pump on the outflow pathway may be excluded. In such an embodiment, both the delivery of therapeutic agent to, and the removal of material from, the osseous tissue may include the use of gravitational flow or siphon action.

In the exemplary osseous delivery system 200, utilized in connection with the treatment of a bone edema, at least initially the outflow volume may exceed the inflow volume. The greater outflow volume may reduce interosseous pressure, as may be associated with bone edema. The greater outflow volume may be experienced for at least a portion of the duration of the osseous delivery procedure. However, as the osseous delivery procedure proceeds, the outflow volume may decrease relative to the inflow volume, for example, as the interosseous pressure is relieved. Accordingly, in some embodiments, at some point during the osseous delivery procedure the outflow volume may be equal to, or even less than, the inflow volume.

Similar to the embodiment disclosed with reference to FIG. 1, the osseous delivery system 200 may provide recirculation of the blood-thinning agent, and/or of other agents delivered to the femur. In such an embodiment, the outflow collection container 216 maybe coupled to the supply 212 of the blood-thinning agent, e.g., via a connective pathway 218, shown in broken line. In one embodiment, recirculation of the blood-thinning agent may be delayed until at least a portion of the fluid producing the interosseous pressure has been removed. The at least partial removal of the fluid producing the interosseous pressure may be determined by sampling and/or analyzing the outflow material, e.g., for the presence of the blood-thinning agent, and/or by other convenient means.

In further embodiments, one or more treatment and/or conditioning modules may be disposed between the outflow pathway and the inflow pathway. Treatment and/or conditioning modules may include, for example, filters that may be provided to remove debris or undesired components from the outflow material prior to reintroduction via the inflow pathway. Other treatment and/or conditioning modules may allow the concentration and/or make-up of therapeutic agents to be adjusted and/or modified. As discussed above, adjustment of the concentration and/or make-up of the therapeutic agent may be based on sampling and/or analysis of the outflow material, etc.

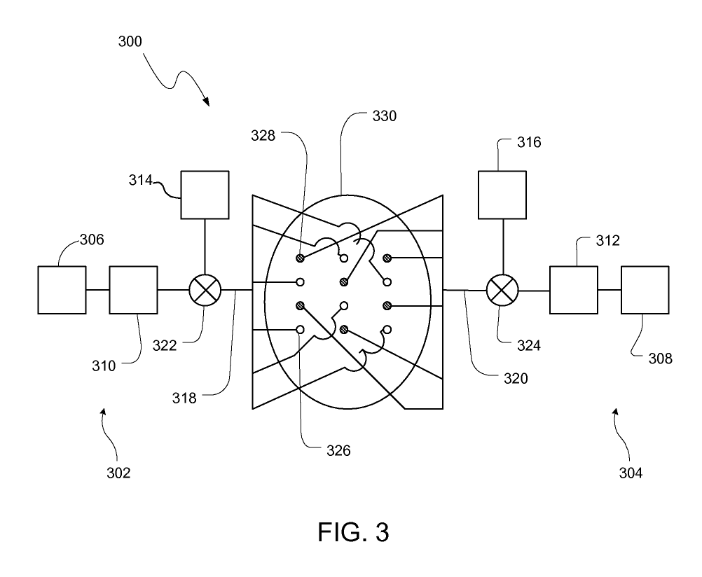

Turning to FIG. 3, an osseous delivery system 300 is shown configured to provide a bi-directional flow of therapeutic agent. The delivery system 300 may include two delivery/recovery systems 302, 304. Each delivery/recovery system 302, 204 may include a delivery pump 306, 308 coupled to a supply reservoir 310, 312, e.g., containing a therapeutic agent, and a recovery pump 314, 316, for example a suction pump. A flow pathway 318, 320 may be selectively coupled to the delivery pump 306, 308 and the recovery pump 314, 316 of each delivery/recovery system 302, 304 by a valve 322, 324. Each flow pathway 318, 320 may include one or more access ports, e.g., 326, 328, providing osseous fluid access.

In the illustrated embodiment, the delivery/recovery systems 302, 304 may be utilized to cool a febrile brain, e.g., by delivery of a cool saline solution, or other therapeutic agents, from the respective reservoirs 310, 312 to the cancellous bone of the skull 330. As shown, the multiple access ports 326, 328 of the respective delivery/recovery systems 302, 304 may provide fluid access to the cancellous bone of the skull 330. For example, the ports 326, 328 may enter the outer cortex and the underlying cancellous bone, although they may not necessarily violate the inner cortex. In an initial state, the valve 322 of the first delivery/recovery system 302 may be configured to permit the first delivery pump 306 to provide a flow of cool saline from the first reservoir 310 to the first flow path. The valve 324 of the second delivery/recovery system 304 may be configured to fluidly couple the second flow pathway 320 to the second recovery pump 316. In this configuration, the first delivery/recovery system 302 may deliver cool saline to the skull 330 via the first plurality of access ports 326 and the second delivery/recovery system 304 may remove at least a portion of the cool saline from the skull 330 through the second plurality of access ports 328 as the saline diffuses, or otherwise migrates, through the cancellous bone of the skull 330. The osseous delivery system 300 may, therefore, provide a cooling flow of saline through the skull 330.

After a period of time the flow of cool saline through the cancellous bone of the skull 330 may be reversed. Reversal of the direction of flow may be achieved by configuring the valve 322 of the first delivery/recovery system 302 to fluidly couple the recovery pump 314 to the first flow pathway 318. Correspondingly, the valve 324 of the second delivery/recovery system 304 may be configured to permit cool saline to be delivered from the second reservoir 312 by the pump 308. In this manner the chilling effect may be increased. Subsequent reversals of the flow direction may be accomplished in a corresponding manner. Rather than merely providing a reversal of the flow direction of cool saline, the reversal of the flow direction may provide the delivery of a different therapeutic agent from the second delivery/recovery system

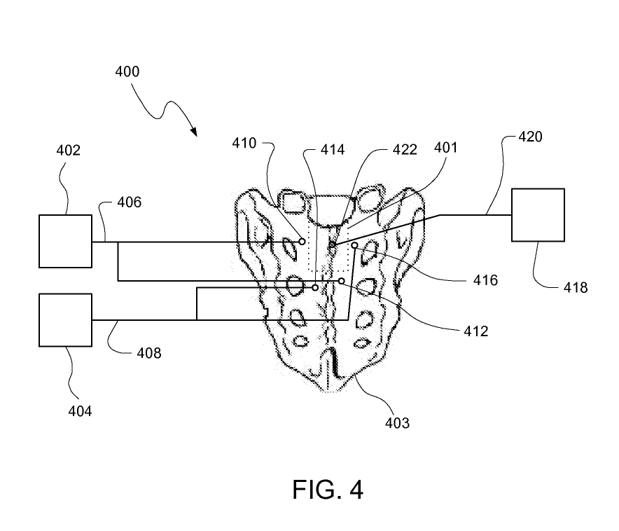

Referring to FIG. 4, an osseous delivery system 400 may be employed for the delivery of a plurality of therapeutic agents through separate inflow pathways. As shown, the system 400 may include supplies 402, 404, e.g., reservoirs, etc., of a first and second component of a two part cement, e.g., for treating a fracture 401 of a sacrum 403. Fluid pathways 406, 408 may provide fluid communication between the supplies 402, 404 of the cement components and a plurality of respective access ports 410, 412, 414, 416. A recovery pump 418, such as a suction pump, may be coupled to an outflow pathway 420 and corresponding outflow access port 422.

The components of the two part cement may be delivered into the sacrum 403 by the suction generated by the recovery pump 418. Additionally, the supplied 402, 404 of the cement components may be positioned so that the delivery of the cement components may be assisted by gravity flow, i.e., the supplied 402, 404 maybe positioned above the sacrum 403.

The outflow access port 422 may be located relative to the fracture 401 and the inflow access ports 410, 412, 414, 416 to encourage the migration of the cement components toward the outflow access port 422 and into the region of the fracture 401. Advantageously, the migration of the cement components may cause the components to at least partially mix. Mixing of the cement components may permit the components to react in situ to produce solid bone cement. The in situ formation of solid bone cement may mend, or at least facilitate mending of, the fracture 401. Any excess cement components may, in some instances, be removed via the outflow pathway. Cement components removed through the outflow pathway may be collected in a collection reservoir, etc. (not shown).

While the cement components may be delivered into the sacrum via gravity flow, which may also be assisted by an outflow pump, the cement components may also be pumped into the sacrum. A delivery pump maybe associated with one or both of the cement components for delivering component into the bone. An outflow pathway, which may include an outflow pump, may facilitate migration of the cement components into the sacrum and mixing of the components therein.

In alternative related embodiments, the foregoing system may be employed to provide the simultaneous or sequential delivery of more than one therapeutic agent through separate access ports. The plurality of therapeutic agents may react in situ within the bone, e.g., to provide a third, reaction, component, as with the multi-component cement. However, the foregoing system may also merely provide the delivery of multiple therapeutic agents via discrete access ports. A common outflow pathway, utilizing one or more access ports, may at least in part direct the migration of the individual therapeutic agents generally toward one or more locations.

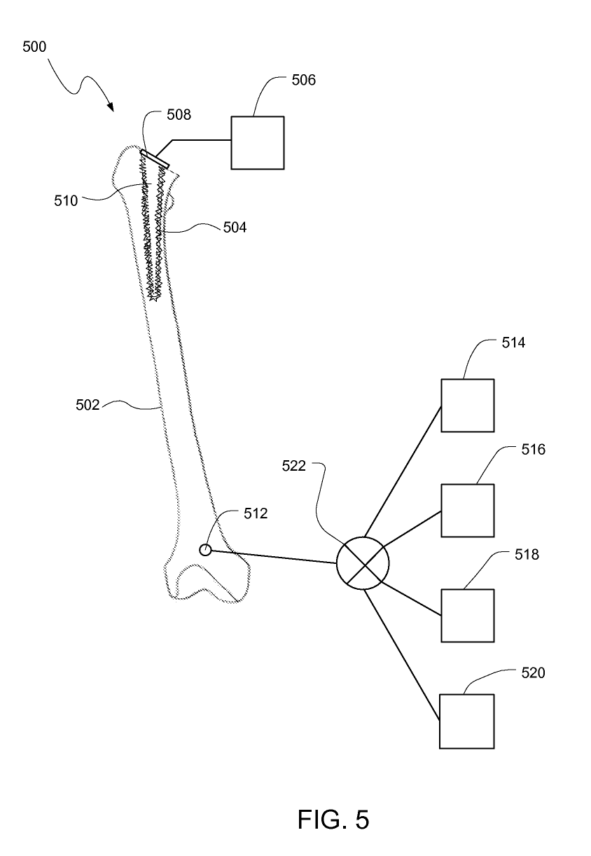

Referring to FIG. 5, another embodiment of an osseous delivery system 500 is depicted. The osseous delivery system 500 may provide serial delivery of multiple therapeutic agents. In the illustrated embodiment, the osseous delivery system 500 may be used in connection with the revision of a hip replacement system. Revision of a hip replacement system may include removal of a primary hip replacement stem (not shown), which may leave the femur 502 with a cement mantle 504, e.g., which may have previously bonded the hip replacement stem in position within the femur 502.

A recovery pump 506, e.g., a suction or vacuum pump, maybe disposed in the region of the opening 508 in the femur 502 remaining after the removal of the hip replacement stem. For example, the recovery pump may be coupled over the opening 508 and may be configured to provide suction drawing from the recess 510 remaining after the removal of the hip replacement stem. At least one inflow access port 512 may be located distal to the cement mantle 504. The inflow access port 512 maybe coupled to a plurality of supplies 514, 516, 518, 520 of therapeutic agents. As shown, multiple therapeutic agents may be delivery through the same inflow access port 512. A selector valve 522 may sequentially couple the plurality of supplies 514, 516, 518, 520 to the inflow access port 512.

In one embodiment, the therapeutic agents may the sequentially delivered to the femur. For example, an agent to dissolve the cement mantle 504 may be provided from the first supply 514. A flushing agent from the second supply 516 may then be delivered to remove any debris, e.g., wear debris or other particulate matter. An antibiotic may be delivered from the third supply 518. Finally, a cement for retaining a revision hip stem may be delivered from a fourth supply 520. The cement may be a one part cement, a two part cement with a second cement component applied to the recess wall or delivered through another route, such as an additional inflow access port. Alternatively, the cement delivered from the fourth supply 520 may interact with the revision implant stem or a coating of the revision implant stem. Various additional and alternative therapeutic agents may also be employed in connection with such an embodiment.

In related embodiments, one or more delivery pumps may be associated with the inflow access port or the individual supplies of therapeutic agents. The delivery pump may facilitate the transport of the therapeutic agents into the osseous matter. Similarly, the supplies of therapeutic agents may be elevated relative to the bone to provide gravity assisted flow of the therapeutic agents into the bone. Additionally, the various supplies of therapeutic agent may be coupled to the inflow access port by a mixing valve, rather than a selector valve. The mixing valve may allow two or more of the therapeutic agents to be delivered simultaneously, rather than sequentially.

In the illustrated embodiments the cannulas of the inflow and outflow pathways are shown positioned adjacent to a feature or region to be treated. However, one or both of the cannulas may be positioned more remotely from the feature or region to be treated, relying instead on diffusion and/or migration of the therapeutic agent through the osseous tissue for delivery to the feature or region to be treated. Consistent with the present disclosure, a therapeutic agent may travel through the osseous tissue via open trabecular structure of bone, naturally occurring passages through the osseous tissue, or through passages created in the osseous tissue, e.g., passages created by drilling, cutting, etc.

The described embodiments herein have contemplated the flow of therapeutic agent from the inflow pathway through an osseous tissue and to the outflow pathway. However, according to various alternative embodiments, the outflow material may be unrelated to the therapeutic agent delivered via the inflow pathway. As such, a flow of therapeutic agent and/or a carrier for a therapeutic agent between the inflow pathway and the outflow pathway is not necessary in the context of the present disclosure.

According to one aspect of the present disclosure, a system and method may provide for the delivery of therapeutic agents to osseous tissue, such as bone or cartilaginous tissue, e.g., intervertebral discs, cartilage, etc., via an inflow pathway. Therapeutic agents consistent with the present disclosure may include any solid, liquid, or gaseous agent which may be delivered during the course of a therapeutic and/or diagnostic procedure. Exemplary therapeutic agents may include non-body temperature saline, anti-tumor therapy agents, anti-coagulant therapies, cement, bone growth therapies, growth plate stunting therapies, demineralization therapies, cells or extra-cellular matrix, anti-biotic therapies, etc. Additionally, the therapeutic agent may include one or more carriers, which may facilitate delivery and/or transport of agents to and/or through the osseous tissue. The therapeutic agent may travel and/or diffuse through at least a portion of the osseous tissue, i.e., bone, cartilaginous tissue, etc., via trabecular structure, passages created in the osseous tissue, etc.

According to another aspect, at least a portion of the volume of therapeutic agent delivered to the osseous tissue may be offset by a volume of material removed from the osseous tissue via an outflow pathway. As such, a system consistent with this aspect may include two or more pathways providing an inflow pathway and an outflow pathway. Additional pathways may provide additional inflow and/or outflow pathways for increasing a delivery and/or removal rate and/or for delivering more than one therapeutic agent, etc. For example, a two component cement may be delivered via two inflow pathways, with one component being delivered through each inflow pathway, for mixture within the osseous tissue. In various embodiments the volume of the inflow of therapeutic agent may be equal to, greater than, and/or less than the volume of material removed from the osseous tissue via the outflow pathway.

Inflow and/or outflow pathways may include portals. The portals associated with the inflow and outflow pathways may include passages and/or openings through the outer layer of osseous tissue to the internal substance and/or structure of the osseous tissue. Portals may be provided by existing holes in the osseous tissue, e.g., arterial foramen. Alternatively, portals may include holes drilled, punched, tapped, etc., through the outer layer of the osseous tissue. Various additional techniques may also be employed for creating portal for providing inflow and outflow into and out of the osseous tissue. As alluded to above, inflow and/or flow portals may be fluidly connected by open trabecular structures of the osseous tissue or through other natural passageways through the osseous tissue. Alternatively, passages may be formed through the osseous tissue, e.g., by drilling, cutting, etc., to connect the portals. Additionally, the inflow and/or outflow pathways may include cannulas, etc., inserted at least partially into the portals to provide connection of the inflow and/or outflow pathways to the osseous tissue.

Delivery of the therapeutic agent and/or removal of material from the osseous tissue may include the use of one or more pumps to deliver the therapeutic agent to the osseous tissue and/or to facilitate the removal of material from the osseous tissue. Accordingly, a pump may be associated with the inflow pathway and/or the outflow pathway. According to related embodiments, the forced delivery of the therapeutic agent to the osseous tissue, e.g., using a pump, may facilitate removal of material from the osseous tissue. In part, the removal of material may be facilitated by the equalization of interosseous pressure. Similarly, the forced removal of material form the osseous tissue, e.g., using a suction pump, may facilitate delivery of the therapeutic agent into the osseous tissue. In further embodiments, gravitational delivery and/or removal may be employed, either alone or in conjunction with a delivery and/or removal pump.

In addition to the use of a pump, an osseous delivery system herein may include the use of one or more reservoirs. The reservoirs may provide a supply of therapeutic agent and/or may allow collection of the material removed from the osseous tissue. Additionally, filtration, sampling, and/or analysis may be carried out on the material removed from the osseous tissue. In one embodiment, the make-up and/or concentration of the therapeutic agent delivered to the osseous tissue may be varied at least in part based on an analysis of the material removed from the osseous tissue.

According to yet another aspect, an osseous delivery system and/or method consistent with the present disclosure may include the use of closed loop and/or semi-closed loop operation. According to this aspect, at least a portion of the material removed from the osseous tissue through the outflow pathway may subsequently be redelivered to the osseous tissue through the inflow pathway. Consistent with a previous aspect, the material removed form the osseous tissue may be filtered or otherwise treated prior to redelivery. Additionally, the material removed from the osseous tissue may be analyzed for the content and concentration of various components and/or materials. The material removed from the osseous tissue may be conditioned to alter the composition and/or concentration of the material prior to redelivery to the osseous tissue. For example, the material removed from the osseous tissue may be fortified with the therapeutic agent prior to redelivery to the osseous tissue.

According to a further aspect, an osseous delivery system and/or method consistent with the present disclosure may include the use of techniques and devices that may provide permanent or temporary use of the system and/or methods disclosed herein. According to this aspect, the inflow and outflow pathways may be provided such that the pathways may be temporarily or permanently placed. For example, temporary placement may be understood as those pathways that may be provided for less than about 90 days, including all values and increments therein, such as 60 day, 30 days, etc. Permanent placement may be understood as those pathways that may be provided for greater than 90 days, including all values and increments therein, such as one year, two years, etc.

The invention herein is set forth by way of specific embodiments consistent therewith. The features and advantages of the several disclosed embodiments are susceptible to combination and modification. Additionally, the disclosed embodiments are provided for the purpose of illustration and not of limitation. Accordingly, the present invention should not be limited by the embodiments disclosed herein.

What is claimed is:

1. A system for delivering a therapeutic agent comprising:
an inflow pathway for delivering said therapeutic agent into osseous tissue; and
an outflow pathway for removing material from said osseous tissue.

2. A system according to claim 1, wherein said inflow pathway comprises a portal formed in said osseous tissue.

3. A system according to claim 1, wherein said outflow pathway comprises a portal formed in said osseous tissue.

4. A system according to claim 1, wherein said inflow pathway and said outflow pathway are in communication through said osseous tissue.

5. A system according to claim 4, wherein said inflow pathway and said outflow pathway are in communication via a passageway formed through at least a portion of said osseous tissue between said inflow pathway and said outflow pathway.

6. A system according to claim 1, comprising a pump coupled to said inflow pathway for delivering said therapeutic agent to said osseous tissue.

7. A system according to claim 1, comprising a pump coupled to said outflow pathway for removing material from said osseous tissue.

8. A system according to claim 1, wherein said inflow pathway and said outflow pathway are coupled to provide an at least partially closed-loop system.

9. A method of treatment comprising:
providing an inflow pathway coupled to an osseous tissue,
providing an outflow pathway coupled to said osseous tissue;
delivering a therapeutic agent to said osseous tissue via said inflow pathway; and
removing a material from said osseous tissue via said outflow pathway.

10. A method according to claim 9, wherein said material removed from said osseous tissue comprises said therapeutic agent.

11. A method according to claim 9, wherein delivering said therapeutic agent to said osseous tissue comprises pumping said therapeutic agent into said osseous tissue.

12. A method according to claim 9, wherein removing said material from said osseous tissue comprises pumping said material out of said osseous tissue.

13. A method according to claim 9, further comprising delivering at least a portion of said material removed from said osseous tissue back to said osseous tissue via said inflow pathway.

14. A method according to claim 13, wherein at least a portion of said material removed from said osseous tissue comprises said therapeutic agent.

15. A method according to claim 13, further comprising conditioning said material removed from said osseous tissue.

16. A method according to claim 9, further comprising a passage extending through at least a portion of said osseous tissue between said inflow pathway and said outflow pathway.

17. An apparatus comprising:
a supply of therapeutic agent;
an inflow pathway coupled to said supply of therapeutic agent, said inflow pathway configured to be fluidly coupled to osseous tissue for delivery of said therapeutic agent to said osseous tissue; and
an outflow pathway configured to be coupled to said osseous tissue for the removal of material from said osseous tissue.

18. An apparatus according to claim 17, further comprising a pump coupled to one of said inflow pathway and said outflow pathway.

19. An apparatus according to claim 17, wherein said inflow pathway comprises a cannula configured to be received in a first portal formed in said osseous tissue.

20. An apparatus according to claim 17, wherein said outflow pathway comprises a cannula figured to be received in a second portal formed in said osseous tissue.

21. An apparatus according to claim 17 wherein said outflow pathway is coupled to said inflow pathway to deliver at least a portion of said material removed from said osseous tissue to said inflow pathway.
